(19) United States
(12) Patent Application Publication
(10) Pub. No.: US 2010/0114131 A1
(43) Pub. Date: May 6, 2010

(54) SKIN TAG REMOVAL SYSTEM

(76) Inventor: ADAM ROTUNDA, Irvine, CA (US)

Correspondence Address: STETINA BRUNDA GARRED & BRUCKER, 75 ENTERPRISE, SUITE 250, ALISO VIEJO, CA, 92656, US

(21) Appl. No.: 12/265,119

(22) Filed: Nov. 5, 2008

(51) Int. Cl. A61B 17/08

(52) U.S. Cl. 606/157

(57) ABSTRACT

A skin tag removal device operative to remove skin tags via the use of compressive force to stop or restrict blood flow to the skin tag. The device comprises first and second members that are attached to one another, preferably via a hinge, and operative to assume an open configuration whereby the members may be positioned about the base of a skin tag and a second closed configuration whereby the first and second members cooperate to impart a compressive force about the base of the skin tag to stop or substantially restrict blood flow. The first and second members may be formed such that the same are biased to one another to impart the compressive force, or may be attached via a hinge and operative to be locked to one another to assume the closed, blood-restricting configuration.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

1. Technical Field of the Invention

The present invention relates generally to a device for removing skin tags protruding from an area of skin and, more particularly, to a skin tag removal device for imparting a compressive force to the base of the skin tag to reduce and/or restrict blood flow to the skin tag for a period of time sufficient to cause necrosis and removal of the skin tag.

2. Description of the Related Art

Skin tags, also known as acrochordons, are small benign tumors that form on an area of skin. Typically, the skin tag protrudes from the skin surface as a bulbous skin mass, and may appear throughout the body including the neck, armpit, groin, face, eyelids. Skin tags vary in size but are usually between 1 mm and 5 cm. The surface of the skin tag may be smooth or irregular in appearance, and often the skin tag is raised from the surface of the skin on a fleshy stalk called a peduncle, which is commonly referred to as the base of the skin tag. Microscopically, the skin tag consists of a fibrovascular core, covered by an unremarkable epidermis.

Although skin tags are harmless, they are frequently removed, usually for cosmetic reasons or due to irritation caused by contact with clothing or jewelry. Skin tags may also interfere with shaving and other grooming rituals. Among the well known methods that may be used to remove skin tags include cauterization with an electrolysis instrument, cryosurgery, ligation, and excision. Typically these procedures are performed at a doctor's office, and may involve the use of a local anesthetic and subsequent freezing with liquid nitrogen, burning by way of a high-frequency electric current or excision using a scalpel or scissors.

Visiting a doctor's office for such a minor procedure involving a harmless skin condition, however, may be costly, time consuming and is otherwise a wasteful use of medical personnel and resources. Accordingly, attempts have been made to develop systems for skin tag removal without the need to visit a doctor's office. In this regard, examples of such devices include adhesive systems, such as those disclosed in pending US Patent Application Publication Number US 2007/0276353 A1, entitled “Device for Removing Acrochordons”, filed Jun. 5, 2007 and published on Nov. 29, 2007, the teachings of which are expressly incorporated herein by reference. Another method for removing skin tags that may be suitable for home use is ligation, which involves tying a thread or string around the base of the skin tag to reduce or restrict blood flow from reaching the skin tag. Lacking sufficient blood flow, the living tissue in the skin tag quickly recedes and falls off spontaneously.

However, some of these methods that allow individuals to remove their skin tags may be difficult to use, expensive, or slow. In addition, individuals seeking to remove skin tags on their own may unnecessarily hurt themselves and cause damage to surrounding areas of the skin due to incompetent or unreliable deployment of a skin tag removal modality. Accordingly, there exists a substantial need in the art for an improved skin tag removal device that is not only fast and effective in removing skin tags, but can be done so easily and without the use of medical personnel, and much less requiring any type of office based procedure. There is a further need in the art for such a system that is exceptionally easy to deploy and can reliably and reproducibly effectuate skin tag removal in a pain free manner that is likewise relatively inexpensive.

### BRIEF SUMMARY

The present invention specifically addresses and alleviates the above-identified deficiencies in the art. In this regard, there is provided a device for removing skin tags that enables self-treatment of benign, easily diagnosed skin tags which appear as small filamentary growths protruding from the skin surface. The skin tag removal device comprises a clip-like structure that is comprised at least a first and second member that are operative to facilitate the application of a compressive force or pressure to a base of the skin tag. When the compressive force is applied to the base of the skin tag the blood flow required to sustain the skin tag is reduced or restricted which initiates necrosis of the skin tag. The sustained application of compressive force to the base of the skin tag is applied for a period of time sufficient to allow the release of the necrotic skin tag from the skin area.

In a first embodiment, the skin tag removal device comprises first and second elongate members connected to one another via a hinge and are operatively transitionable between a first open configuration whereby the first and second elongate members may be separated from one another, and a second compressive configuration whereby the first and second elongate members are caused to compress inwardly toward one another. To that end, the first and second members may be joined by a living hinge that biases the first and second members inwardly toward one another, or may otherwise be joined by a joint, pivot or other similar-type mechanism that enables the first and second members to operatively transition between the first and second configurations.

When the clip of the skin tag removal device is oriented in the open position, the first and the second member may be placed in close proximity to the base of the skin tag. In other words, the open position of the clip enables the base of the skin tag to be received. The opening between the first member and the second member associated with the open position may be adjusted in accordance with the size of the skin tag. Because both the first member and the second member are movable relative to each other, the skin tag removal device may accommodate skin tags of many different shapes and sizes by moving either the first member or the second member accordingly. When in the open position, the first member and the second member may form an opening similar to an open jaw for the base of the skin tag to be received. From the open position, the first member and the second member may move toward each other to steadily decreasing the opening between the first member and the second member, resembling a closing scissor. The opening may decrease until the closed position is achieved. As the first member and the second member move closer to each other a compressive force is imparted onto the base of the skin tag. The compressive force stops reduces the blood flow to the skin tag by reducing circulation at the base of the skin tag.

As discussed above, the first member and the second member may be joined to one another by a hinge. The hinge may facilitate the pivotal movement of the first member relative to the second member. The hinge may also facilitate the pivotal movement of the second member relative to the first member. As a result, the hinge connecting the first member and the second member enables the clip to transition between the open and closed positions. The hinge may further define a fixed axis of rotation and both the first member and the second member may be capable of rotating about the fixed axis of motion. The rotation of the first member and the second member relative to the fixed axis of rotation allows for the opening to be increased or decreased dependent upon whether the clip is to be oriented in the open or closed position. To remove the skin tag, the first member and the second member may move to the open position. Depending on the size of the skin tag and in particular the base of the skin tag, the opening between the first and second members may be adjusted to receive the base of the skin tag. Subsequent to receiving the base of the skin tag, the first member and the second member may move toward each other causing the opening between the two members to decrease whereby the opening is reduced until the closed position is attained. As the opening between the two members is reduced, the compressive force applied to the base of the skin tag is increased until the closed position between the first member and the second member is achieved.

In the closed clip position, the first member and the second member of the skin tag removal device may be secured to each other such that the closed position may be maintained until the necrotic skin tag is released from the skin surface to which it was attached. To maintain the closed position between the first member and the second member of the skin tag removal device a mechanical fastener may be used. Preferably, the mechanical fastener used to secure the ends of the first member and the second member is located opposite the hinge at which the first and second members are joined such that when the first member and the second member are locked together the application of compressive force from the clip to the base of the skin tag is maintained. This allows for removal of the skin tag following a brief period of time in which the first member and the second member cooperate to impart a compressive force to stop or substantially reduce blood flow to the skin tag.

The mechanical fastener used to secure the first member and the second member in the closed clip position may be a latch that may include a hook and a complementary rib that are configured to secure the first member and the second member in the closed position. For example, the first member may include a hook extending from a distal end. The second member may include a rib extending from its distal end. As the first member and the second member move from the open position to the closed position, the hook may receive the rib. The first member and the second member may be locked in place and may be prevented from moving to the open position without a user's release of the latch. The rib may be unlatched from the hook when the closed position is no longer desirable. For example, after the skin tag is removed, the mechanical fastener may be unlatched.

The mechanical fastener used to secure the first member and the second member in the closed clip position may alternatively be a screw-type lock that secures the first member to the second member in the closed clip position. The screw lock includes a screw and an aperture for receiving the screw. The first member may include the screw disposed within the distal end, and the second member may include the aperture for receiving the screw. In the open position, the screw disposed within the first member may not be in contact with the aperture of the second member or alternatively, the screw may be loosely fitted within the aperture when the clip is in the open position. As the opening between the first member and the second member is reduced, the screw may be threaded into the aperture of the second member to securely couple the first member to the second member when the clip is in the closed position. After receiving the base of the skin tag, the first member and the second member may be moved to the closed position and the screw may then be received into the aperture. The screw and the aperture compliment each other. The opening between the first member and the second member is reduced when the screw is threaded into the aperture and consequently the first member and the second member impart a compressive force on the base of the skin tag. The screw being threaded into the aperture prevents the first member and the second member from disengaging and thus maintaining the closed position which is maintained until the skin tag is removed from the skin surface.

In a second embodiment of the skin tag removal device, a clip having a base member may be provided. The clip may be configured to be operatively transitional between an open position and a closed position. The skin tag removal device includes a base member having a first end and a second end. A first member is pivotally coupled to the first end of the base member and a second member is pivotally coupled to the second end of the base member whereby the first member and the second member are biased toward one another. A biasing force enables the first member and the second member to intersect when the clip is oriented in the closed position. Both the first member and the second member may be moved opposite a biasing force configured to increase the spatial separation between the first and the second member. For example, the first member may be moved or pushed away from the second member while the second member is being pushed away from the first member. Preferably, the first member and the second member may each include a knob or other surface that enables a user to separate the first member and the second member with his or her fingertips to thus facilitate the separation of the first member and the second member and attain the open position. The open position is configured to enable the device to receive the base of the skin tag. To move from the open position to the closed position, the force used to separate the first member and the second member is released or reduced to allow the first member and the second member to return to the closed position in accordance with the biasing force. As the first member and the second member move toward the closed position, a compressive force is applied to the base of the skin tag. The biasing force ensures that the first and the second member maintain the closed position and thus consistently apply the compressive force from the clip to the base of the skin tag to stop or reduce blood flow to the skin tag sufficient to cause necrosis in the skin tag.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of a skin tag located on an area of skin;

FIG. 2 is a perspective view of a skin tag removal device in an open position being positioned about a skin tag;

FIG. 3 is a perspective view of the skin tag removal device in a closed position;

FIG. 4 is a side elevational view of the skin tag removal device in the closed position and locked by a latch;

FIG. 5 is a side elevational view of the skin tag removal device in the closed position and locked by a screw lock;

FIG. 6 is a perspective view of the skin tag removal device in accordance with a second embodiment wherein a first member and a second member are biased toward each other;

FIG. 7 is a perspective view of the skin tag removal device in accordance with the second embodiment wherein the first member and the second member are separated opposite a biasing force; and

FIG. 8 is a perspective view of the skin tag removal device returning to the biased position.

### DETAILED DESCRIPTION

The above description is given by way of example, and not limitation. Given the above disclosure, one skilled in the art could devise variations that are within the scope and spirit of the invention disclosed herein, including various ways of devising a skin tag removal device configured to be oriented in a closed position and an open position. Further, the various features of the embodiments disclosed herein can be used alone, or in varying combinations with each other and are not intended to be limited to the specific combination described herein. Thus, the scope of the claims is not to be limited by the illustrated embodiments.

The device of the present invention allows for the removal of skin tags using a pair of members used to press against the base of the skin tag to reduce or restrict the blood flow to the skin tag. The different components form part of a device which may be placed adjacent to the skin, and in particular around a base of the skin tag to thus cut off incoming blood flow. The members are configured to apply or impart a pressure to achieve that end.

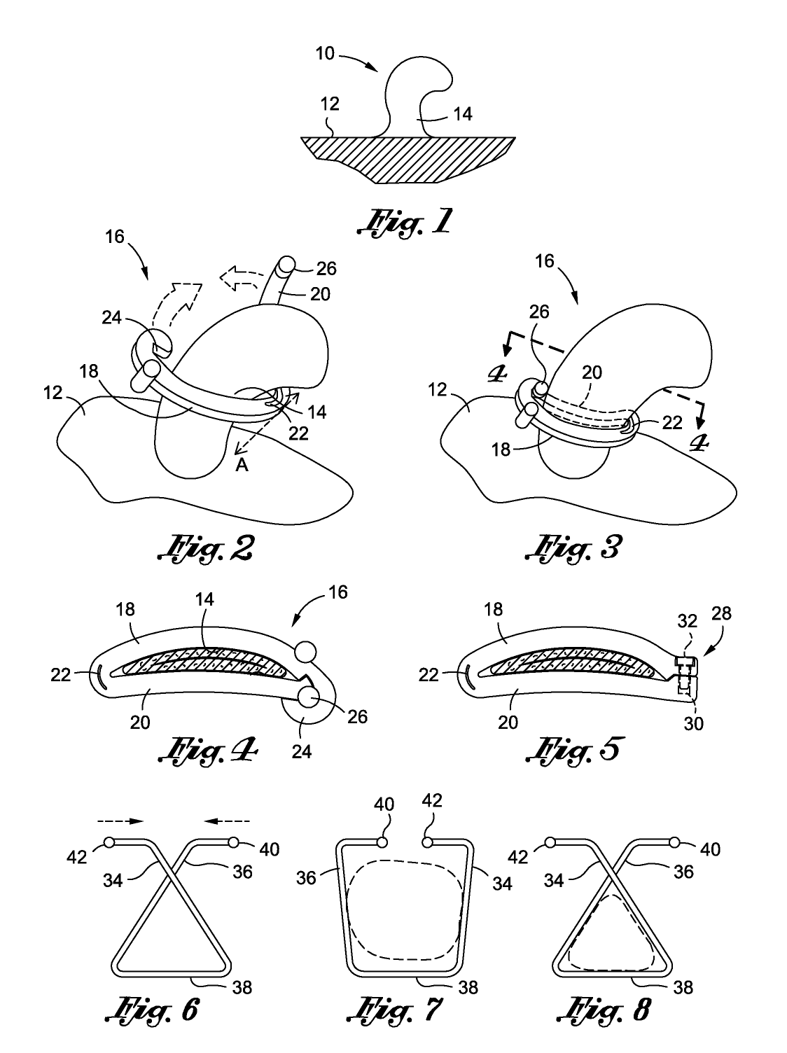

As shown in FIG. 1, a skin tag 10 protrudes from a surface of skin 12. The skin tag 10 includes a base 14 protruding from the surface of skin 12 and receiving blood flow. As is well-known, removal of the skin tag 10 from the surface of skin 12 may be achieved by restricting the blood flow to the skin tag 10. After a period of time has elapsed in which the blood flow to the skin tag 10 is restricted, the skin tag 10 may simply fall of the surface of skin 12. A skin tag is usually less than 10 mm and has a diameter at the base on the skin which is less than 2 mm, which means that very little compression pressure is required to occlude the venous or the arterial blood flow in the capillary blood circulation located in the periphery.

FIG. 2 provides a skin tag removal device 16 that may be used to reduce blood flow to the skin tag 10 and subsequently separate or remove the skin tag from the surface of skin 12. The skin tag removal device 16 includes a first member 18 and a second member 20 joined to one another. The members 18, 20 of the device 16 are configured to move relative to one another, and may be joined to one another by a hinge 22. The hinge 22 may define a joint where the first member 18 and the second member 20 are pivotally coupled to each other. The hinge 22 also defines a fixed axis of rotation A. The first member 18 and the second member 20 are configured to rotate about the fixed axis of rotation A. As shown by the arrows in FIG. 2, the first member 18 may rotate about the fixed axis of rotation A so that the first member 18 may move toward or away from the second member 20. The second member 20 may likewise rotate about the fixed axis of rotation A to move toward or away from the first member 18.

The first member 18 and the second member 20 of the skin tag removal device 16 are configured to move away from each other to be oriented in a first open position. The open position is like an open jaw and is configured to receive the skin tag 10. In this regard, the first member 18 and the second member 20 may be placed adjacent the skin surface 12 from where the skin tag 10 protrudes. The open position or open jaw orientation assumed by the removal device 16 as it is placed against the skin surface 12 allows for the skin tag 10 to protrude through the open space between the first member 18 and the second member 20. The device 16 may then be aligned so that the base 14 of the skin tag 10 may rest against the first member 18, the second member 20 or both the first member 18 and the second member 20. Because the first member 18 and the second member 20 are rotatable about the fixed axis of rotation A, the space defined between both the first member 18 and the second member 20 is adjustable and may be adjusted depending upon the size of the skin tag 10. For example, if the skins tag 10 is larger than the average size of skin tags, the first member 18 and the second member 20 may be rotated so that the space between the first member 18 and the second member 20 is increased. The increased space between the two connected members 18 and 20 allow for the skin tag 10 to be received.

Referring now to FIGS. 2 and 3, the first member 18 and the second member 20 may rotate about the fixed axis of rotation A associated with the hinge 22 towards each other. As both the first member 18 and the second member 20 rotate towards each other the space between the first member 18 and the second member 20 is reduced until the device 16 is oriented in a second closed position. As the first member 18 and the second member 20 move toward each other from the open position to the closed position, the compressive force imparted onto the base 14 of the skin tag 10 is increased. The compressive force applied to the base 14 of the skin tag 10 by the first member 18 and the second member 20 stops or substantially reduces the blood flow to the skin tag 10. Subsequent to the reduction of blood flow to the skin tag 10, the skin tag 10 may detach from the surface of skin 12.

Referring now to FIGS. 4 and 5, the skin tag removal device 16 may be locked in the closed position. The advantage of locking the first member 18 and the second member 20 in the closed position is the ability to maintain the compressive force on the base 14 of the skin tag 10 until the skin tag 10 is detached from the surface of skin 12. To achieve that end, the distal end of the first member 18 may include a hook 24 and the distal end of the second member 26 may include a rib 26 that is complementary to the hook 24. In this regard, as the first member 18 and the second member 20 move toward the closed position the hook 24 may be fitted around the rib 26 to secure the first member 18 to the second member 20 and thus assumed a locked configuration.

In another embodiment, the skin tag removal device 16 may be secured in the closed position by a screw lock 28. The screw lock 28 implementation of the skin tag removal device 16 may be used for skin tags that are larger than average size skin tags. The larger the skin tag the greater the compressive force necessary to impede blood flow and ultimately detach the skin tag 10 from the surface of skin 12. In this scenario it may not be practical to use a latch similar to the hook 24 and the rib 26 and the screw lock 28 may provide stronger security of the first member 18 and the second member 20. The screw lock 28 may be comprised of two different elements including a screw 32 and an aperture 30 for receiving the screw 32. The screw 32 may be disposed within the distal end of the first member 18 and the aperture 30 for receiving the screw 32 may be located on the distal end of the second member 20. As a result, when the first member 18 and the second member 20 are moved from the open position to the closed position, the screw 32 may be received by the aperture 30. The screw may include threads on the screw surface so that the screw 32 may be fastened to the aperture 30 to hold the distal end of the first member 18 together with the distal end of the second member 20. This enables the first member 18 and the second member 20 to remain secure in the closed position to reduce blood flow to the skin tag 10. This may cause the skin tag 10 to detach or fall off from the skin surface 12.

Referring now to FIGS. 5-8 and initially to FIG. 6, a second embodiment of the skin tag removal device is provided. In this embodiment, the first member 34 may be pivotally coupled to a first end of a base member 38. The second member 36 may be pivotally coupled to a second end of the base member 38. The first member 34 and the second member 36 member are biased toward one another. In this regard, the first member 34 and the second member 36 intersect to define a closed position associated with the skin tag removal device. Both the first member 34 and the second member 36 may include a nob 40, 42 for receiving a force opposite the biasing force that causes the first member 34 and the second member 36 to intersect. A user of the skin tag removal device may push the nobs 40, 42 opposite the biasing force until the first member 34 and the second member 36 no longer intersect as shown in FIG. 7. The nobs 40 and 42 may be pushed to enable an open position defined by the first member 34 and the second member 36.

In the open position, the skin tag removal device may receive the skin tag 10. In particular, the base member 38, the first member 34, and the second member 36 may be placed adjacent the skin surface 12 to surround the base 14 of the skin tag 10. After the skin tag 10 is received, the skin tag removal device may be returned to its original position as provided in FIG. 8. In other words, the force from the user exerted on the nobs 40, 42 of the first member 34 and the second member 36 is removed or reduced so that the biasing force allows the first member 34 and the second member 36 to intersect and return to the closed position. Upon returning to the closed position, the base 14 of the skin tag 10 is pinched by the first member 34 and the second member 36. The biasing force allows the first member 34 and the second member 36 to consistently pinch the base 14 of the skin tag 10. Pinching the base 14 of the skin tag 10 reduces blood flow and, after a sufficient period of time in which the blood flow is reduced or restricted from reaching the skin tag 10, the skin tag 10 may detach from the surface of skin 12.

Additional modifications and improvements of the present invention may also be apparent to those of ordinary skill in the art. Thus, the particular combination of parts and steps described and illustrated herein are intended to represent only certain embodiments of the present invention, and is not intended to serve as limitations of alternative devices and methods within the spirit and scope of the invention. For example, it should be understood that the device of the present invention can be configured to have a generally U-shape or V-shape such that the first and second members are joined by an integrally formed living or spring-like hinge such that the first and second members are inwardly biased towards one another and can impart a compressive force about the base of a skin tag. In such embodiments, the U-shaped or V-shaped devices are manipulated such that first and second members are manually spread apart so that the same may be received about the base of the skin tag and thereafter allowed to clamp about the base of the skin tag, which ultimately stops or restricts blood flow. Along these lines, it should be understood that for any of the embodiments discussed herein, such devices may be fabricated from any of a variety of materials well-suited to accomplish the objectives of the present invention, and in particular to impart a constant, compressive force about the base of a skin tag, and thus can include any of a variety of wire or metal type materials, plastics and the like.

What is claimed is:

1. A device for removing skin tags, comprising:
a clip configured to be oriented in an open position and a closed position and including:
a first member and a second member joined to one another, the first member and the second member being pivotally coupled to each other for moving between the open position and the closed position, the open position being operative to receive a base of a skin tag and the closed position being operative to impart a compressive force to the base of the skin tag to reduce blood flow to the skin tag.

2. The device of claim 1, wherein the first member and the second member are joined to one another by a hinge, the hinge defining a fixed axis of rotation, the first member configured to rotate about the fixed axis of rotation relative to the second member, the second member configured to rotate about the fixed axis of rotation relative to the first member.

3. The device of claim 1, wherein the first member and the second member are secured to each other in the closed position by a mechanical fastener.

4. The device of claim 3, wherein the mechanical fastener is a latch.

5. The device of claim 3, wherein the mechanical fastener is a screw lock.

6. A device for removing skin tags, comprising:
a clip configured to be oriented in an open position and a closed position and including:
a base member having a first end and a second end;
a first member pivotally coupled to the first end of the base member;
a second member pivotally coupled to the second end of the base member;
wherein the first member and the second member are biased toward each other by a biasing force when the clip is oriented in the closed position, the first member and the second member configured to move opposite the biasing force when the clip is oriented in the open position so that the skin tag is received by the clip, the first member and the second member imparting a compressive force on the skin tag to reduce blood flow when the clip returns to the closed position in response to the biasing force.

7. A device for removing skin tags, comprising:
a clip configured to be oriented in an open position and a closed position and including:
a first member and a second member attached to each other by a hinge, the hinge defining a fixed axis of rotation, the first member being configured to rotate about the fixed axis of rotation relative to the second member, the second member being configured to rotate about the fixed axis of rotation relative to the second member;
the first member and the second member being operative to rotate to the open position for receiving the skin tag, the first member and the second member being operative to rotate to the closed position to impart a compressive force on the skin tag for reducing blood flow to the skin tag.
